(19) United States
(12) Patent Application Publication
(10) Pub. No.: US 2020/0166491 A1
(43) Pub. Date: May 28, 2020

(54) TESTING METHOD TO REVEAL SWEAT TRANSFER FROM SKIN TO FABRICS

(71) Applicant: L'OREAL, Paris (FR)

(72) Inventors: Amanda Rezende MIRANDA, Rio de Janeiro (BR); Thiago FERREIRA GOMES, Rio de Janeiro (BR); Daphine Antonia CLEMENTE, Rio de Janeiro (BR)

(21) Appl. No.: 16/612,049

(22) PCT Filed: May 10, 2017
(86) PCT No.: PCT/BR2017/050110

§ 371 Date: Nov. 8, 2019

(51) Int. Cl. G01N 31/22; G01N 21/78; G01N 33/36

(52) U.S. Cl. CPC G01N 31/222; G01N 33/367; G01N 21/78

(57) ABSTRACT

A method of assessing efficacy of an amine-free antiperspirant is provided. The method comprises (a) contacting a textile with skin having sweat, wherein the sweat is formed on the skin having the antiperspirant thereon, whereby the sweat is transferred from the skin onto the textile, (b) contacting the textile from step (a) with an effective amount of a solution for generating a purple color on the textile, wherein the solution comprises ninhydrin and a solvent, whereby a purple color is generated on the textile, and (c) determining an intensity of the purple color on the textile, wherein a high intensity of the purple color indicates a low efficacy of the antiperspirant.

### FIELD OF THE INVENTION

The invention relates to a method of assessing efficacy of an antiperspirant.

### BACKGROUND OF THE INVENTION

An antiperspirant is commonly used to prevent or reduce perspiration or sweat formation on skin to which the antiperspirant has been applied. In general, people are not able to see sweat marks on clothes, but notice a bad smell after several hours because bacteria digest odorless sweat and produce volatile compounds with a bad smell. The bad odor on clothes may be produced from sweat on skin and then transferred to the clothes, or produced from sweat on the clothes after the sweat is transferred from skin to the clothes.

An antiperspirant contains ingredients that carry out antiperspirant effects. Methods have been developed to assess or evaluate the potential efficacy of an antiperspirant. For example, WO 2016/193167 describes a testing method of demonstrating the potential for an antiperspirant effect of a cosmetic composition based on the detection of a visible color change generated by contacting the cosmetic composition with a detector substance for aluminum compounds, which are used in many cosmetic compositions to provide antiperspirant effects. However, such a testing method does not provide direct assessment of an antiperspirant for its efficacy because the testing method does not measure any effect that the antiperspirant may have on sweat formed on skin to which the antiperspirant has been applied.

Ninhydrin (2,2-dihydroxyindane-1,3-dione) reacts with primary amines, particularly from amino acids, to form a deep blue or purple color compound known as Ruhemann's purple (diketohydrindylidenediketohydrindamine). Fingerprints consist of approximately 98% water and the remaining 2% is a combination of grease, oil, salts and amino acids. Ninhydrin has been used in the chemical forensic field to visualize latent fingerprints on a substrate based on the formation of the Ruhemann's purple after contacting the substrate with ninhydrin. Some problems are associated with the development of latent fingerprints with ninhydrin. For example, the ability to develop latent fingerprints on a substrate depends on the type of substrate. On most substrates, latent fingerprints are not readily visible. It may take as long as three weeks to develop latent fingerprints on thermal papers using conventional ninhydrin methods.

Thus, there remains a need for a simple and direct test of efficacy of an antiperspirant.

### SUMMARY OF THE INVENTION

The invention provides a method of assessing efficacy of an amine-free antiperspirant. The method comprises (a) contacting a textile with skin having sweat, wherein the sweat is formed on the skin having the antiperspirant thereon, whereby the sweat is transferred from the skin onto the textile, (b) contacting the textile from step (a) with an effective amount of a solution for generating a purple color on the textile, wherein the solution comprises ninhydrin and a solvent, whereby a purple color is generated on the textile, and (c) determining an intensity of the purple color on the textile, wherein a high intensity of the purple color indicates a low efficacy of the antiperspirant. The antiperspirant may be in the form of a stick, aerosol, roll-on, wipe or cream. The purple color may become visible no more than 30 minutes after contacting the textile with the solution. The method may further comprise comparing the intensity of the purple color determined in step (c) with an intensity of a control purple color, wherein the control purple color is generated by contacting the solution with a textile having sweat formed on and transferred from skin not having the antiperspirant thereon, or skin having a control antiperspirant thereon.

The solution may comprise 0.5-2 wt % ninhydrin. For example, the solution may comprise 1.0 wt % or 1.3 wt % ninhydrin.

The solvent may be volatile. For example, the solvent may be ethanol or methanol.

In step (b), the solution may be sprayed onto the textile.

Steps (b) and (c) may be carried out at a temperature in a range from 15° C. to 50° C. For example, steps (b) and (c) may be carried out at 20° C. or 45° C. Preferably, steps (b) and (c) are carried out at room temperature.

### BRIEF DESCRIPTION OF THE DRAWINGS

FIG. 1 shows efficacy of an antiperspirant in Volunteer No. 1. Significant reduction in color intensity was observed on a textile sample having sweat transferred from the left armpit (top panels) or right armpit (bottom panels) to which an antiperspirant (right panels) had previously been applied, as compared with a textile sample having sweat transferred from the corresponding armpit to which a deodorant (left panels) had previously been applied.

FIG. 2 shows efficacy of an antiperspirant in Volunteer No. 2. Significant reduction in color intensity was observed on a textile sample having sweat transferred from the left armpit (top panels) or right armpit (bottom panels) to which an antiperspirant (right panels) had previously been applied, as compared with a textile sample having sweat transferred from the corresponding armpit to which a deodorant (left panels) had previously been applied.

FIG. 3 shows efficacy of an antiperspirant in Volunteer No. 3. Significant reduction in color intensity was observed on a textile sample having sweat transferred from the left armpit (top panels), but not right armpit (bottom panels), to which an antiperspirant (right panels) had previously been applied, as compared with a textile sample having sweat transferred from the corresponding armpit to which a deodorant (left panels) had previously been applied.

FIG. 4 shows efficacy of an antiperspirant in Volunteer No. 4. Significant reduction in color intensity was observed on a textile sample having sweat transferred from right armpit (bottom panels), but not the left armpit (top panels), to which an antiperspirant (right panels) had previously been applied, as compared with a textile sample having sweat transferred from the corresponding armpit to which a deodorant (left panels) had previously been applied.

FIG. 5 shows efficacy of an antiperspirant in Volunteer No. 5. Significant reduction in color intensity was observed on a textile sample having sweat transferred from the left armpit (top panels) or right armpit (bottom panels), to which an antiperspirant (right panels) had previously been applied, as compared with a textile sample having sweat transferred from the corresponding armpit to which a deodorant (left panels) had previously been applied.

FIG. 6 shows efficacy of an antiperspirant in Volunteer No. 6. Significant reduction in color intensity was observed on a textile sample having sweat transferred from the left armpit (top panels), but not right armpit (bottom panels), to which an antiperspirant (right panels) had previously been applied, as compared with a textile sample having sweat transferred from the corresponding armpit to which a deodorant (left panels) had previously been applied.

FIG. 7 shows 4 points marked on a piece of fabric onto which a dosed jet was applied in Example 1.

### DETAILED DESCRIPTION OF THE INVENTION

The present invention provides a method of assessing efficacy of an antiperspirant. The inventors have discovered that sweat transferred from skin onto a textile can be visualized by exposing the textile to ninhydrin such that an antiperspiration benefit of a product can be clearly seen. This method is simple, quick and sensitive to detect a change in an amount of sweat formed on skin after an antiperspirant has been applied to the skin. This method may be developed into a standard protocol to obtain reproducible results in testing antiperspirant efficacy of products.

The term “antiperspirant” used herein refers to a cosmetic composition comprising ingredients that control perspiration and body odor. An antiperspirant can prevent sweat from reaching the skin surface and reduce bacteria that cause body odor. An antiperspirant is applied to skin, for example, of an armpit, to reduce or prevent sweat formation on the skin. An amine-free antiperspirant does not contain an amine group that reacts with ninhydrin to generate a color change, for example, a purple color. An antiperspirant may be in the form of a stick, aerosol (or spray), roll-on, wipe or cream. The term “deodorant” used herein refers to a cosmetic composition comprising ingredients that control body odor, but not perspiration. The term “efficacy of an antiperspirant” used herein refers to the effectiveness of an antiperspirant in controlling perspiration or sweat formation on skin. Preferably, the skin is of an individual who needs to control perspiration.

The terms “textile,” “fabric” and “cloth” are used herein interchangeably and refer to a flexible material made from natural or artificial fibers such as yarns or threads.

According to the present invention, a method of assessing efficacy of an amine-free antiperspirant is provided. The method comprises (a) contacting a textile with skin having sweat, which is formed on the skin having the antiperspirant thereon, whereby the sweat is transferred from the skin onto the textile, (b) contacting the textile from step (a) with an effective amount of a solution for generating a purple color on the textile, wherein the solution comprises ninhydrin and a solvent, whereby a purple color is generated on the textile, and (c) determining an intensity of the purple color on the textile, wherein a high intensity of the purple color indicates a low efficacy of the antiperspirant. This method may not include heating.

In step (a), the textile may be contacted with the skin for a time period from about 1 minute to about 24 hours. For example, the textile may be contacted with the skin for at least or no more than about 1, 5, 10, 20, 30, 40 or 50 minutes or about 1, 2, 3, 4, 5, 6, 7, 8, 9, 10, 12, 18 or 24 hours.

In a preferred embodiment, after the sweat is transferred onto the textile, the textile having the sweat is dried before being contacted with the solution comprising ninhydrin and a solvent.

The term “an effective amount” refers to an amount of the solution comprising ninhydrin required to achieve a stated goal. The effective amount of the ninhydrin solution may be selected to generate a purple color on a textile. The effective amount of the ninhydrin solution may vary depending upon the amount of the sweat on the textile, the composition of the textile, the ninhydrin concentration of the solution, the nature of the solvent in the solution, and the means used to contact the textile with the ninhydrin solution. A specific effective amount for a given textile having sweat may generally be set by the judgment of a technician.

The ninhydrin solution may comprise about 0.5-2 wt % ninhydrin. For example, the ninhydrin solution may comprise about 1.0 or 1.3 wt % ninhydrin. Unless stated otherwise, all wt % figures herein are relative to the total composition.

The solvent of the ninhydrin solution may be any volatile solvent in which ninhydrin is soluble. Preferably, the solvent is colorless. The solvent may be an alcohol such as ethanol or methanol.

In step (b), contacting the textile with the ninhydrin solution may be accomplished by any means. For example, the ninhydrin solution may be sprayed onto the textile for a predetermined period of time, for example, from about 1 second to about 180 minutes, for about 1, 5, 15, 30 or 45 seconds, or about 1, 5, 10, 30, 60, 120 or 180 minutes), or until the textile is wet. Alternatively, the textile may be soaked in the ninhydrin solution for a predetermined period of time, for example, from about 1 second to about 180 minutes, or for about 1, 5, 15, 30 or 45 seconds, or about 1, 5, 10, 30, 60, 120 or 180 minutes.

Step (b) may be carried out under conditions permitting the generation of a purple color. Step (b) may be carried out at a temperature in a range from about 15° C. to about 50° C., for example, at about 20° C. or about 45° C. Preferably, step (b) is carried out at room temperature. Step (b) may be carried out under a humidity in a range of about 10%-90% or about 20%-60%, or less than about 70% or about 80%.

The purple color may become detectable no more than about 1, 5, 10, 15, 30, 45 or 60 minutes after contacting the textile with the solution. The purple color may become visible no more than about 1, 5, 10, 15, 30, 45 or 60 minutes after contacting the textile with the solution.

Step (c) may be carried out at a temperature in a range from about 15° C. to about 50° C., for example, at about 20° C. or about 45° C. Preferably, step (c) is carried out at room temperature. Step (c) may be carried out under a humidity in a range of about 10%-90% or about 20%-60%, or less than about 70% or about 80%. In some embodiments, steps (b) and (c) are carried out under the same conditions, for example, at the same temperature and humidity.

The method according to the present invention may be used to compare the efficacy of two antiperspirant products by carrying out each step of the method under the same conditions. For example, the textiles are in contact with skin having sweat under the same temperature and humidity for the same time period after the antiperspirant products are applied two different pieces of skin at comparable locations (e.g., left and right armpits) on the same person; the same amount of ninhydrin is applied to the textiles; and the intensity of the purple color generated on the textiles is determined under the same temperature and humidity at the same time after applying ninhydrin to the textiles.

The method may further comprise comparing the intensity of the purple color determined in step (c) with an intensity of a control purple color. The control purple color is generated by contacting the solution with a textile having sweat formed on and transferred from skin not having the antiperspirant thereon or skin having a control antiperspirant thereon. The relative intensity of the purple color generated on textile having sweat formed on and transferred from skin having an antiperspirant thereon as compared with the control purple color indicates the relative efficacy of the antiperspirant. This comparison test may be used as a standard test to obtain reproducible efficacy results for new antiperspirants.

### Example 1

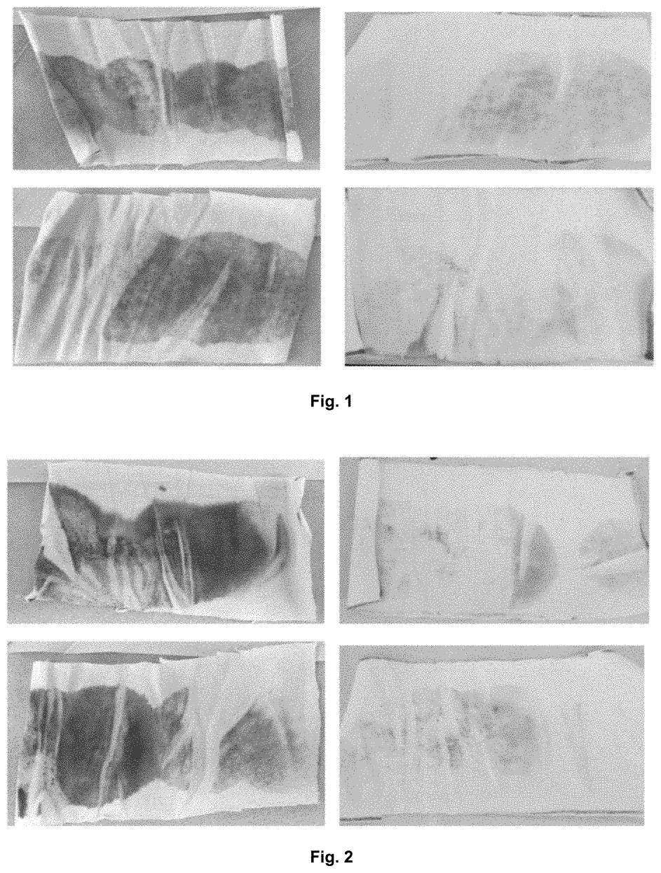

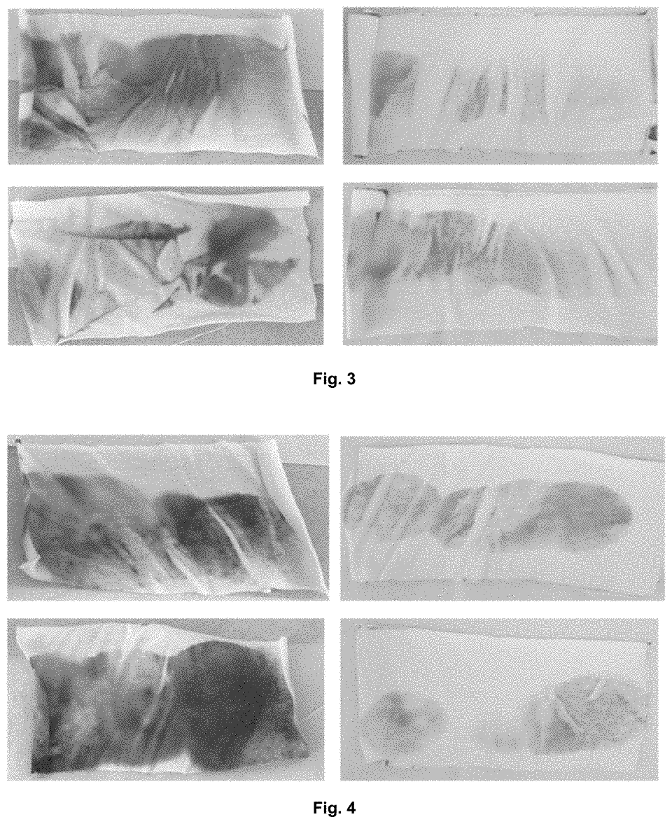

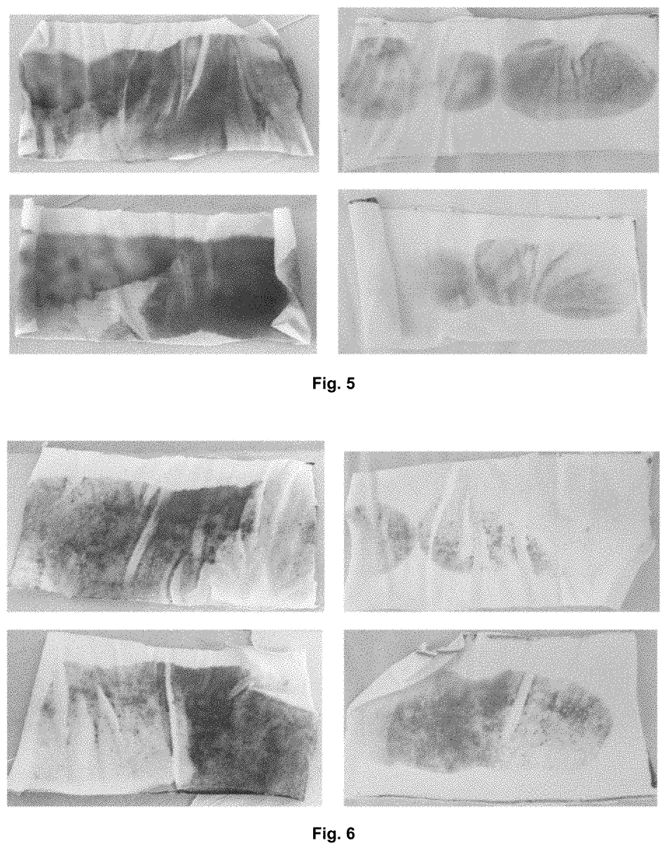

A study was carried out to compare visualization of sweat transferred to textile samples from armpits of 7 volunteers who had previously applied deodorants and antiperspirants to their armpits. After applying deodorants or antiperspirants, the volunteers stayed in a sauna at 32° C. for 10 minutes before removing and placing each textile sample in a plastic bag. Under a chemical hood, the textile samples were sprayed with 1% ninhydrin solution. After 30 minutes, the textile samples were examined for visible purple color. One volunteer did not have enough sweat in the deodorant experiment, and thus did not proceed with the antiperspirant experiment. Significant reduction in purple color intensity was observed on 9 of 12 textile samples having sweat transferred from armpits to which antiperspirants had previously been applied, as compared with textile samples having sweat transferred from corresponding armpits to which deodorants had previously been applied (FIGS. 1-6). Below is the protocol.

1. Reagents and Materials:

a. T-shirts washed with soap and water;

b. Absolute Alcohol;

c. 1% Ninhydrin solution;

d. Dosing pump;

e. Plastic binder bags.

2. Preparation of 1% alcoholic ninhydrin solution:

a. Weigh 1 g of ninhydrin PA and solubilize in 100 g of alcohol;

b. Transfer the solution to the dosing pump.

3. Procedure 1 (Performed in the sauna)

a. Cut rectangular pieces of fabric at 100 cm² (evaluation support) from pre-washed shirts (10 min);

b. Place one piece of the fabric under each armpit of the volunteers for 10 min. Note: Procedure performed with alcohol-sanitized gloves to avoid contamination;

c. Remove and place the fabric in an identified binder bag. Avoid folding fabrics. Between one armpit and another, always clean the glove with alcohol to avoid contamination.

4. Procedure 2 (Performed in the chemical hood)

a. Clean the area of the chemical hood with alcohol. Place a contact adhesive over it;

b. Place the piece of textile in the demarcated area;

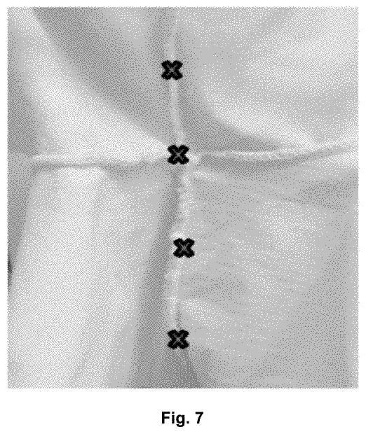

c. Apply a dosed jet (0.5 g±0.02) of the ninhydrin solution at 4 points marked in the cut (FIG. 7);

d. Check the color intensity and the spot size after 30 minutes of reaction.

The term “about” as used herein when referring to a measurable value such as an amount, a percentage, and the like, is meant to encompass variations of ±20% or ±10%, more preferably ±5%, even more preferably ±1%, and still more preferably ±0.1% from the specified value, as such variations are appropriate.

Although the invention is illustrated and described herein with reference to specific embodiments, the invention is not intended to be limited to the details shown. Rather, various modifications may be made in the details within the scope and range of equivalents of the claims without departing from the invention.

1. A method of assessing efficacy of an amine-free antiperspirant, comprising:
(a) contacting a textile with skin having sweat, wherein the sweat is formed on the skin having the antiperspirant thereon, whereby the sweat is transferred from the skin onto the textile;
(b) contacting the textile from step (a) with an effective amount of a solution for generating a purple color on the textile, wherein the solution comprises ninhydrin and a solvent, whereby a purple color is generated on the textile; and
(c) determining an intensity of the purple color on the textile, wherein a high intensity of the purple color indicates a low efficacy of the antiperspirant.

2. The method of claim 1, further comprising comparing the intensity of the purple color determined in step (c) with an intensity of a control purple color, wherein the control purple color is generated by contacting the solution with a textile having sweat formed on and transferred from skin not having the antiperspirant thereon or skin having a control antiperspirant thereon.

3. The method of claim 1, wherein the solution comprises 0.5-2 wt % ninhydrin.

4. The method of claim 1, wherein the solvent is volatile.

5. The method of claim 4, wherein the solvent is ethanol.

6. The method of claim 4, wherein the solvent is methanol.

7. The method of claim 1, wherein the solution is sprayed onto the textile.

8. The method of claim 1, wherein steps (b) and (c) are carried out at a temperature from 15° C. to 50° C.

9. The method of claim 1, wherein steps (b) and (c) are carried out at room temperature.

10. The method of claim 1, wherein the purple color becomes visible no more than 30 minutes after contacting the textile with the solution.

11. The method of claim 1, wherein the antiperspirant is in the form of a stick, aerosol, roll-on, wipe or cream.
